(19) United States
(12) Patent Application Publication
(10) Pub. No.: US 2013/0202654 A1
(43) Pub. Date: Aug. 8, 2013

(54) Topical Foam Composition

(71) Applicants: Amar Lulla, Mumbai (IN); Geena Malhotra, Mumbai (IN); Shrinivas Madhukar Purandare, Mumbai (IN)

(72) Inventors: Amar Lulla, Mumbai (IN); Geena Malhotra, Mumbai (IN); Shrinivas Madhukar Purandare, Mumbai (IN)

(73) Assignee: CIPLA LIMITED, Mumbai (IN)

(21) Appl. No.: 13/511,303

(22) PCT Filed: Nov. 23, 2010
(86) PCT No.: PCT/GB2010/002161

§ 371 Date: Dec. 21, 2012

(30) Foreign Application Priority Data
Nov. 23, 2009 (IN) 2703/MUM/2009
Sep. 13, 2010 (IN) 2522/MUM/2010

(51) Int. Cl. A61K 9/12; A61K 45/06; A61M 35/00; A61K 31/437

(52) U.S. Cl. CPC A61K 9/122; A61K 31/437; A61K 45/06; A61M 35/003
USPC 424/400; 514/279; 424/45; 514/161; 514/150; 514/171; 604/310

(57) ABSTRACT

A topical foam pharmaceutical composition for rectal administration comprising rifaximin in the form of nanosized particles is described. Also described is a method of making the composition and the use of the composition to as a medicament.

### FIELD OF INVENTION

The present invention relates to a topical foam composition of rifaximin suitable for rectal administration, its process of manufacturing and its use for the treatment, prophylaxis, or maintenance of remission of colonic, anal or rectal dysfunction.

### BACKGROUND AND PRIOR ART

Anal disorders including anal fissure, anal ulcer, and acute haemorrhoidal disease and benign conditions of the anal canal, are common amongst the subjects of all ages, races and sexes. However, these conditions can be problematic to treat and inconvenient if not painful to endure. A subject with an anal fissure or ulcer frequently experiences anal pain and bleeding, the pain being more pronounced during and after bowel movements.

Haemorrhoids are specialized vascular areas lying subjacent to the anal mucosa.

Various therapies have been devised to treat these anal disorders. Typical, non-surgical therapy includes bulk laxatives and sitz baths. Sitz baths are helpful because they induce relaxation of the anal sphincter mechanism. (Shafik, “Role of warm-water bath in anorectal conditions: The thermosphincteric reflex,” Journal of Clinical Gastroenterology., 16:304-308, 1993).

Topical anal therapy is also used as one of the approaches to promote healing, relieve pain, and reduce swelling and inflammation. Many preparations have been tried including those containing local anesthetics, corticosteroids, astringents, antibiotics and other agents.

Although administration via the peroral route is the most commonly targeted goal of new drug and dosage form research and development, oral administration is not always feasible or desirable. The potential for oral dosage form development is severely limited for active agents that are poorly absorbed in the upper gastrointestinal (GI) tract and unstable to proteolytic enzymes. Some agents cause local stomach or upper GI irritation or require doses in excess of 500 mg. Certain patient populations, notably children, the elderly, and those with swallowing problems, are often difficult to treat with oral tablets and capsules. Additionally, treatment of some diseases is best achieved by direct administration near the affected area, particularly with diseases involving anorectal tissues. Although oral administration can be used for drugs targeted for some of these diseased tissues, exposure of the entire body compartment to the administered drug is inefficient and can lead to undesired adverse effects.

Rectal drug administration is amenable, however, to both local and systemic drug delivery. It has been effectively utilized to treat local diseases of the anorectal area as well as to deliver drugs systemically as an alternative to oral administration. Some advantages of this targeted delivery which includes large surface area, ability to bypass first-pass metabolism, prolonged residence time makes this route more promising for delivery of locally acting drugs.

Suppositories, solutions, suspensions, or retention enemas represent some of the rectal dosage forms. Of these, liquid preparations have very limited application, largely due to inconvenience of use and poor patient compliance. Semi-solid preparations like gels, foams or ointments for rectal administration can afford advantages over liquid formulations because retention of the dosage form in the rectal cavity reduces patient compliance problems.

However, none of the formulations available have been convincingly shown to reduce the healing time or to reliably ameliorate associated pain.

Treatments, such as with Neosporin® ointment (which contains three antibiotics Neomycin, Polymyxin B Sulfate and Bacitracin Zinc), are very sensitizing. Hence, there is still a need in the art to provide compositions useful to reduce the healing time, which alleviate pain and promote healing of the affected rectal and anal tissues.

Rifaximin is a water insoluble semi-synthetic rifamycin-based non-systemic antibiotic belonging to the rifamycin class of antibiotics, and has the scientific name [(2S,16Z,18E,20S,21S,22R,23R,24R,25S,26S,27S,28E)-5,6,21,23,25-pentahydroxy-27-methoxy-2,4,11,16,20,22,24,26-octamethyl-2,7-(epoxypentadeca-[1,11,13]trienimino)benzofuro[4,5-e]pyrido[1,2-a]-benzimida-zole-1,15(2H)-dione,25-acetate]. Rifaximin has the following chemical formula.

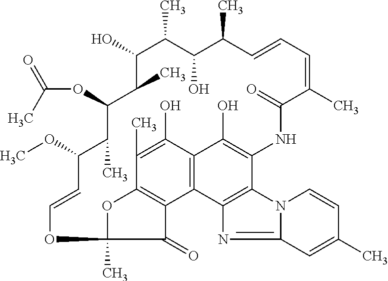

Rifaximin has been described to be endowed with an antibacterial activity similar to the activity of rifampin [Venturini A. P. and Marchi E., Chemiotherapia, 5 (4), 257-256, (1986)]. However, its mechanism of action differs from rifampin in that it is not absorbed through the systemic route after oral administration [Venturini A. P., Chemotherapy, 29, 1-3, (1983) and Cellai L. et al., Chemiotherapia, 3, (6), 373-377, (1984)] due to the zwitterionic nature of the compound, which cannot be absorbed by the gastrointestinal tract [Marchi E. et al., Journal of Medicinal Chemistry., 28, 960-963, (1985)].

Rifaximin is soluble in alcohol, ethyl acetate, chloroform and toluene. It exerts its broad-spectrum antibacterial activity by inhibiting bacterial RNA synthesis in the gastrointestinal tract against localized gastrointestinal bacteria that cause infectious diarrhea, irritable bowel syndrome, small intestinal anal disease, Crohn's disease, and/or pancreatic insufficiency. Rifaximin is licensed by the U.S. Food and Drug Administration to treat traveler's diarrhea caused by E. coli.

Rifaximin has low systemic absorption with Cₘₐₓ of 3.4 ng/mL, Tₘₐₓ of 0.8 hours and is moderately bound to plasma proteins (67.5%). It has half-life of 1.8 hours and is primarily excreted in feces (97% of administered dose) and 0.32% in the urine.

Rifaximin is not absorbed by the oral route [Venturini A. P., Chemotherapy, 29, 1-3, (1983)] nor by topical application [Venturini A. P. et al., Drugs Under Experimental and Clinical Research., 13, 4, 233-6, (1987)].

Due to this particular pharmacokinetic behavior, rifaximin has no toxicity at a dose of 2000 mg/kg/os, when administered orally in rats, and therefore, on the basis of the microbiological, pharmacodynamic and toxicological data, the drug has been used for the treatment of bacterial gastroenteritis, neurological symptoms and clinical symptoms of hepatic encephalopathy and for the pre- and post-surgical treatment of the gastrointestinal tract [Alvisi V. et al., Journal of International Medical Research., 15, 49-56, (1987), Testa R. et al., Drugs under Experimental and Clinical Research., 11, 387-392, (1985), Gruttadauria G. et al., European Review for Medical and Pharmacological Sciences., 9, 100-105, (1987)].

Rifaximin is used for the treatment of pathologies caused by non-invasive strains of Escherichia coli, a micro-organism which is not able to penetrate into GI mucosa and therefore remains in contact with gastrointestinal fluids.

Rifaximin is also approved for the treatment of pathologies whose etiology is in part or totally due to intestinal acute and chronic infections sustained by Gram-positive and Gram-negative bacteria, with diarrhea syndromes, altered intestinal microbial flora, summer diarrhea-like episodes, traveler's diarrhea and enterocolitis; pre- and post-surgery prophylaxis of the infective complications in gastro intestinal surgery; and hyperammonaemia therapy as coadjutant.

Rifaximin is available in tablets, granules for oral suspension and ointment, marketed in Europe and U.S.A. and in many other countries. Tablets, for example are currently marketed at the dosage of 200 mg for traveler's diarrhea under the brand name Xifaxan®.

U.S. Pat. No. 5,352,679 discloses use of rifaximin in formulations for treatment of gastric dyspepsia caused by Helicobacter pylori bacteria. The rifaximin formulations disclosed in the patent are in the form of a tablet (such as a sugar coated tablet), capsule, granules or syrup for oral administration.

U.S. Pat. No. 5,314,904 and U.S. Pat. No. 6,140,355 disclose compositions containing rifaximin for treatment of vaginal infections.

WO2007/103448 discloses pharmaceutical preparations comprising an anti-rectal dysfunction agent and rifaximin. The preferred anti-rectal dysfunction is a nitric oxide modulating agent such as nitroglycerin. The examples disclosed in the patent application are related to the ointment containing rifaximin and nitroglycerine.

WO2004/037225 discloses cosmetic or pharmaceutical foam carrier suitable for inclusion of both water soluble and oil soluble pharmaceutical and cosmetic agents.

EP0468555 and EP0395329 disclose aqueous foam compositions in which the same substance or mixture of substances (namely one or more chlorofluorocarbons) is used as both a foaming agent and a propellant for expulsion of the composition out of a conventional aerosol can.

However, there is little disclosure in prior art about the topical formulations of rifaximin which are capable of providing the desired therapeutic effect.

It is known that topical treatment of infections or disturbances of the colon or rectum is more preferred than oral route, as the formulation is directly applied to the site of action and hence rapidly reaches and acts on the point at which the disturbance is located.

According to the state of the art, topical delivery of active agents is achieved preferably by rectal administration using suppositories, enemas, ointments, creams and foam. Of these the suppository is the most common one. The suppository base is generally a fat-soluble but may also be water-soluble or water-miscible base. To obtain a good bioavailability the active ingredient should come into contact with the rectal or colonic mucosa.

Ointments and creams often do not create an environment for promoting respiration of the wounded tissue and which is not favorable to the normal respiration of the skin. Moreover, there may be likelihood of experiencing pain and irritation during the application of ointments and creams, particularly to abraded, wounded or inflamed mucosa of the rectum or colon.

Aqueous foamable preparations are the less common of the rectal preparation forms. They require relatively complicated manufacture as well as complicated packaging as compared with suppositories and enema. However, since better spreading effects are obtained with enema and foams than with suppositories more distal intestine regions can be reached thereby.

Although the delivery of active ingredient using foam can provide various advantages as compared to the other topical delivery forms such as better spreading in the surrounding tissues, rectal foams are complicated formulations which may not form under arbitrary circumstances because it requires a special balance between the foam-forming components. Slight shifts in the composition may result in collapse of the foam or alternatively the foam is not formed at all, especially when administration is to occur via an applicator nozzle with small diameter. Most foam dosage forms for rectal delivery have incorporated corticosteroids to date, although some products have also been used to deliver antiseptics, antifungal agents, anti-inflammatory agents, local anesthetic agents, skin emollients, and protectants (American Journal of Drug Delivery, 2003, vol. 1 (1), pp. 71-75). However, only a few are commercially available.

Conventional foams for rectal or vaginal administration are filled in pressurised containers with a pharmaceutically active ingredient dissolved or suspended in a liquid vehicle, at least one propellant gas and a surfactant with foaming properties.

Because of the hydrophobic nature of rifaximin, it is virtually insoluble in water but is readily soluble in alcohols. An adequate amount of active substance can be dissolved by the use of solubilizers such as organic solvents, water-soluble alcohols. However the formulations if prepared in this way; may remain stable over a short period because large amounts of the active substance are decomposed within a short time.

Because of this problem, topical rifaximin formulations which can be used directly by the patient in the administration form ready for use have still remained challenging. The suitable compositions of rifaximin suggested in the prior art are ointment and vaginal foam. The ointment is not in the form of ready to use, but can be prepared by a cumbersome process of crushing the rifaximin tablet in suitable oily vehicle and admixing this mixture with ointment base prior to the application. Moreover, the vaginal foam when formulated may also not remain stable when provided in compressed gas packs.

Thus there still exists a need to develop a topical foam composition of rifaximin suitable for rectal administration with increased diffusion, useful in reduction of healing time, alleviate pain and promote healing of the affected rectal and anal tissues and also remains stable during the storage period.

### OBJECT OF THE INVENTION

An object of the present invention is to provide a topical foam composition of rifaximin suitable for rectal administration.

Another object of the present invention is to provide a topical foam composition of rifaximin having better spreading effect.

Yet another object of the present invention is to provide a topical foam composition of rifaximin which remains stable over the storage period.

One more object of the present invention is to provide a process for preparing the topical pharmaceutical composition of rifaximin suitable for rectal administration.

Still another object of the present invention is to provide is to provide a method for treating, prophylaxis, or maintenance of remission of colonic or rectal dysfunction by administering the topical foam composition of rifaximin to patients in need thereof.

A further object of the present invention is to provide a topical foam composition of rifaximin for rectal administration which remains effective even after intestinal evacuation by the subject treated.

### SUMMARY OF THE INVENTION

According to the first aspect of the present invention there is provided a topical foam composition of rifaximin for rectal administration.

According to the second aspect of the present invention there is provided a topical foam composition of rifaximin for rectal administration wherein rifaximin is in the nanosize form.

According to a third aspect of the present invention there is provided a topical foam composition of rifaximin for rectal administration comprising one or more pharmaceutical excipients or carriers.

According to a fourth aspect of the present invention there is provided a process of preparing the said topical foam composition of rifaximin.

According to a fifth aspect of the present invention there is provided a topical foam composition of rifaximin for use in the preparation of a medicament suitable for administering to the rectum, colon and/or terminal ileum of a patient for the treatment, prophylaxis, or maintenance of remission of colonic or rectal dysfunction.

According to sixth aspect there is provided a method of treating, preventing, or alleviating an anal disorder comprising administering the topical foam of rifaximin to a subject in need thereof.

### DETAILED DESCRIPTION OF THE INVENTION

The inventors have developed a topical foam composition of rifaximin which may achieve the aforesaid objectives and which also exhibits a topical anti-infective action.

Surprisingly, the inventors have found that by utilizing nanosized rifaximin it is possible to increase the dispersion of Rifaximin which is suitable for rectal administration.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 00, Number 00, March 2010].

Nanosizing leads to increase in the exposure of surface area of rifaximin particles leading to an increase in the rate of dissolution.

The present invention thus provides a pharmaceutical composition, comprising rifaximin wherein rifaximin is in the nanosize range.

The term “Rifaximin” is used in broad sense to include not only “Rifaximin” per se but also their pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs, pharmaceutically acceptable prodrugs, pharmaceutically acceptable complexes etc.

The nanoparticles of the present invention can be obtained by any of the process such as but not limited to milling, precipitation, homogenization and the like.

The pharmaceutical composition of the present invention comprises rifaximin having an effective particle size range of less than 1000 nm, preferably less than 500 nm. The effective particle size is preferably at least 10 nm. In an embodiment the effective particle size is in the range from 200 to 300 nm. In an embodiment, at least 50% of the rifaximin particles have an effective particle size in the range from 10 to less than 1000 nm. In an embodiment, at least 50% of the particles have an effective particle size in the range 200 to 300 nm.

According to one embodiment of the present invention, the process of milling comprises dispersing rifaximin particles in a liquid dispersion medium in which rifaximin is poorly soluble, followed by applying mechanical means in the presence of grinding media to reduce the particle size of rifaximin to the desired effective average particle size.

According to another embodiment of the present invention, the process of precipitation comprises dissolving rifaximin in a suitable solvent, adding the dissolved rifaximin to a solution comprising at least one surface stabilizer; and causing precipitation by using an appropriate non-solvent.

According to another embodiment of the present invention, the process of homogenization comprises dispersing rifaximin particles in a liquid dispersion medium, followed by subjecting the dispersion to homogenization to reduce the particle size of the rifaximin to the desired effective average particle size.

According to another embodiment of the present invention, the process of high pressure homogenization comprises rifaximin presuspension (containing rifaximin in the micrometer range) by subjecting the rifaximin to air jet milling in the presence of an aqueous surfactant solution. The presuspension is then subjected to high-pressure homogenization in which it passes through a very small homogenizer gap of about 25 μm which leads to a high streaming velocity. High-pressure homogenization is based on the principle of cavitations (i.e., the formation, growth, and implosive collapse of vapor bubbles in a liquid.

According to another embodiment of the present invention, the process of spray-freeze drying involves the atomization of an aqueous rifaximin solution into a spray chamber filled with a cryogenic liquid (liquid nitrogen) or halocarbon refrigerant such as chlorofluorocarbon or fluorocarbon. The water is removed by sublimation after the liquid droplets solidify.

According to another embodiment of the present invention, the process of supercritical fluid technology involves controlled crystallization of rifaximin from dispersion in supercritical fluids, carbon dioxide.

According to another embodiment of the present invention, the process of double emulsion/solvent evaporation technique involves preparation of oil/water (o/w) emulsions with subsequent removal of the oil phase through evaporation. The emulsions are prepared by emulsifying the organic phase containing rifaximin, polymer and organic solvent in an aqueous solution containing emulsifier. The organic solvent diffuses out of the polymer phase and into the aqueous phase, and is then evaporated, forming rifaximin-loaded polymeric nanoparticles.

According to a further embodiment of the present invention, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of rifaximin ranging from 20 nm to more than 100 μm.

According to a further embodiment of the present invention, the process of thermal condensation involves use of capillary aerosol generator (CAG) to produce high concentration condensation submicron to micron sized aerosols from rifaximin solutions.

According to a further embodiment of the present invention, the process of ultrasonication involves application of ultrasound during particle synthesis or precipitation, which leads to smaller particles of rifaximin and increased size uniformity.

According to a further embodiment of the present invention, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of rifaximin.

According to a preferred embodiment of the present invention, reducing the particle size to a nanosize range involves nanomilling of rifaximin preferably with at least one surface stabilizer, at least one viscosity building agent and at least one polymer to form the nanomilled slurry.

The term “Rifaximin” is used in broad sense to include not only “Rifaximin” per se but also their pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs, pharmaceutically acceptable prodrugs, pharmaceutically acceptable complexes etc.

The amount of rifaximin in the rectal foam composition according to the present invention ranges from about 0.01% w/w to 10% w/w, preferably about 0.5% w/w to about 8% w/w of the total weight of the composition.

The composition preferably contains a vehicle, which is preferably a water soluble alkanol. Water soluble alkanols which are suitable for use in the present invention may be selected from, but not limited to ethanol, polyalcohols such as a propylene glycol, glycerol, polyethylene glycol, polypropylene glycol, propylene glycol glyceryl esters or mixtures thereof.

In particular, by use of a specific ratio of water soluble alkanols to water in topical foam composition comprising rifaximin, it remains stable over the storage period. The ratio of water soluble alkanol to water may range between about 0.05:10 to 10:0.05 on a weight basis.

Thus, in an embodiment, the present invention provides a topical foam composition comprising rifaximin wherein rifaximin is in the nanosize range with one or more pharmaceutical excipient/carrier in a suitable dosage form for rectal delivery

In another embodiment of the present invention the nanomilled Rifaximin is provided as a rectal foam filled in a compressed gas container, which upon valve actuation, emits a fine dispersion of liquid and/or solid materials in a gaseous medium. The said composition is easier to apply, less dense, and spread more easily than other topical dosage forms.

Alternatively, the composition may be formulated in various ways to provide emollient or drying functions to the rectal mucosa, depending on the formulation constituents.

Another benefit of the pharmaceutical composition of the present invention is ease of use by the patient and consumer acceptance.

In a preferred embodiment, the topical foam composition of the present invention comprises rifaximin wherein rifaximin is in the nanosize range, at least one surfactant and at least one propellant, water soluble alkanols, water and optionally any other pharmaceutical excipients or carriers.

Although mixture of water soluble alkanols is preferred vehicle for the topical non-aqueous foam composition according to the present invention, suitable non-aqueous vehicle which may be employed in the topical foam composition of the invention, which include but are not limited to stearyl alcohol, glyceryl monoricinoleate, glyceryl monostearate, propane-1,2-diol, butane-1,3-diol, mink oil, cetyl alcohol, ispropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polythylene glycol, triethylene glycol, lanolin, sesame oil, coconut oil, arachis oil, sunflower seed oil, evening primrose oil, castor oil, lanolin alcohols, petrolatum, mineral oil, butyl myristate, isostearic acid, palmitic acid, isopropyl linoleate, lauryl lactate, myristyl lactate, decyl oleate, myristyl myristate, ethyl alcohol, methylene chloride, isopropanol, castor oil, ethylene glycol monoethyl ether, diethylene glycol monobutyl ether, diethylene glycol monoethyl ether, dimethyl sulphoxide, dimethyl formamide, tetrahydrofuran, glycerin, sorbitol, sodium 2-pyrrolidone-5-carboxylate, soluble collagen, dibutyl phthalate, soaps and fatty alcohols.

It is also desirable to use a suitable vehicle which is compatible with the rectal and colonic mucosa.

Alternatively, the liquid vehicle may also be based on highly hydrophilic organic substances to allow the surfactant to perform its foaming action, which however must not be inhibited by the other substances present in the formulation, such as the active principles, stabilizers, whereas the specific adjuvants (such as foam consistency correctors) must be chosen from those with strong hydrophilic and lipophilic characteristics.

The vehicle typically constitutes from 10% w/w to 95% w/w, preferably from 10% w/w to 90% w/w, more preferably from 20% to 70% w/w relative to the total weight of the composition.

In a preferred embodiment, the vehicle employed in the topical foam composition of the present invention comprises water in an amount from approximately 20% w/w to approximately 90% w/w relative to the total weight of the composition and a water-soluble alkanol, preferably propylene glycol, in an amount from approximately 20% w/w to 50% w/w relative to the total weight of the composition. Preferably, the vehicle contains 20-80% w/w water relative to the total weight of the composition. Preferably the vehicle contains 5-40% w/w water soluble alkanol relative to the total weight of the composition. Most preferably, the vehicle contains 20-80% w/w water relative to the total weight of the composition, and 5-40% w/w water soluble alkanol relative to the total weight of the composition.

The preferred amount of non-aqueous vehicle, especially the water soluble alkanol, more especially the propylene glycol, is from 10% to 40% w/w based on the total weight of the composition.

Surface active agents which may be employed in the aqueous foam composition of the present invention include, but are not limited to anionic surfactants, non-ionic surfactants, cationic surfactants, and amphoteric surfactants.

Anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, diammonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof.

Nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethylene oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Amphoteric surfactants include, but are not limited to, sodium N-dodecyl-alanine, sodium N-lauryl-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquatemium, stearalkonium chloride, sterayl dimethylbenzyl ammonium chloride, stearyl trimethyl ammonium chloride, trimethylglycine, and combinations thereof. The preferred amount of surfactant, is present in an amount from 0.1% to 10.0% w/w based on the total weight of the composition; more preferably, in an amount from 0.1% to 8.0% w/w based on the total weight of the composition.

It will be appreciated by the person skilled in the art that amongst the two or more surfactants selected; at least one surfactant selected may provide the emulsifying action whereas the other may provide a foam-stabilizing action. The surfactant(s) is desirably chosen are such that it remains compatible with the rectal and colonic mucosa and will be present in an amount which achieves the desired pharmaceutical effect but which does not give rise to problems of irritation.

In a further embodiment of the present invention, the topical foam composition contains a lubricant. Preferably, said lubricant is a silicone (e.g. polydimethylsiloxane). The silicone may further stabilize the foam-forming composition.

The propellant used in the topical foam composition of the present invention is used to accomplish the foaming effect. The propellant may be chosen according to known principles for preparing a foamable composition of the aerosol type packed in a pressurized container and suitable for a rectal application. The propellant may be any suitable, pharmaceutically acceptable, gas such as a low molecular weight hydrocarbon e.g. isobutane, n-butane, propane, CFC, hydrocarbons; chlorofluorocarbons (CFCs); hydrochlorofluorocarbons (HCFCs); hydrofluoroalkanes (HFAs) such as HFA 134a and HFA 227; or air. Preferably, the propellant comprises a mixture of n-butane, isobutane, propane.

The propelling properties can vary depending on the type and quantity of propellant used and, consequently, the foam can reach more or less distant regions of the large intestine.

The propellant may be present in an amount from 0.05 to 20% w/w, preferably 0.5 to 20% w/w of the composition. Preferably, said amount is between 3 to 10%, more preferably between 7 to 9% w/w of the composition. Additionally, liquefied nitrogen may be present as a pressurizing agent to obtain the required number of doses.

Further, the topical foam composition according to the present invention may comprise at least one additional active ingredient suitable for rectal administration.

Additional active agents may be may be selected from, but are not limited to one or more anti-inflammatory agents, steroids (e.g. corticosteroids), additional antibiotics, anti-fungal agents, analgesics, or anti-neoplastic agents.

Suitable antibiotics include, but are not limited to, dapsone, chloramphenicol, neomycin, cefaclor, cefadroxil, cephalexin, cephradine, erythromycin, clindamycin, lincomycin, amoxicillin, ampicillin, bacampicillin, carbenicillin, did oxacillin, cyclacillin, picloxacillin, hetacillin, methicillin, nafcillin, penicillin, polymyxin, tetracycline, amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, picloxacillin, hetacillin, methicillin, nafcillin, penicillin, polymyxin or tetracycline.

Suitable anitfungal agents include but are not limited to, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, and others such as acrisorcin, amorolf[iota]ine, biphenamine, bromosalicylchloranilide, buclosamide, calcium propionate, chlophenesin, ciclopirox, cloxyquin, coparaff[iota]nate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, undecylenic acid.

Antifungal agents may also include, polyenes such as amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, acrisorcin, amorolf[iota]ne, biphenamine, bromosalicylchloranilide, buclosamide, calcium propionate, chlophenesin, ciclopirox, cloxyquin, coparaff[iota]nate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion or undecylenic acid.

Other therapeutic agents can include a steroidal or non-steroidal antiinflammatory agent. Non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflum[iota]c acid, tolfenamic acid, diflurisal, flufenisal, piroxicam, sudoxicarn, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof.

Suitable corticosteroids include but are not limited to, hydrocortisone, i.e., 11-17-21-trihydroxypregn-4-ene-3,20-dione or Cortisol, Cortisol acetate, hydrocortisone phosphate, hydrocortisone 21-sodium succinate, hydrocortisone tebutate, corticosterone, corticosterone acetate, cortisone, cortisone acetate, cortisone 21B-cyclopentanepropionate, cortisone phosphate, triamcinolone hexacetonide, dexamethasone phosphate, desonide, betamethasone dipropionate, mometasone furate.

The corticosteroid and topical anesthetic may be employed together in the composition along with rifaximin.

For inflammation, preferred treatments for use in combination therapy with the compositions of the present invention include, but not limited to, naproxen sodium (Anaprox(R) and Anaprox(R) DS, Roche), flurbiprofen (Ansaid(R); Pharmacia), diclofenac sodium+misoprostil (Arthrotec(R), Searle), valdecoxib (Bextra(R), Pharmacia), diclofenac potassium (Cataflam(R) and Voltaren(R), Novartis), celecoxib (Celebrex(R), Pfizer), sulindac (Clinoril(R), Merck), oxaprozin (Daypro(R), Pharmacia), salsalate (Disalcid(R), 3M), difhmisal (Dolobid(R), Merck), naproxen sodium (EC Naprosyn(R), Roche), piroxicam (Feldene(R), Pfizer), indomethacin (Indocin(R) and Indocin SR(R), Merck), etodolac (Lodine(R) and Lodine XL(R), Wyeth), meloxicam (Mobic(R), Boehringer Ingelheim), ibuprofen (Motrin(R), Pharmacia), naproxen (Naprelan(R), Elan), naproxen (Naprosyn(R), Roche), ketoprofen (Orudis(R) and Oruvail(R), Wyeth), nabumetone (Relafen(R), SmithKline), tolmetin sodium (Tolectin(R), McNeil), choline magnesium trisalicylate (Trilisate(R), Purdue Fredrick), and rofecoxib (Vioxx(R), Merck).

Antineoplastic agents may also be included in the topical foam composition of the present invention along with rifaximin which include, but not limited to, vincristine, vinblastine, vindesine, busulfan, chlorambucil, spiroplatin, cisplatin, carboplatin, methotrexate, adriamycin, mitomycin, bleomycin, cytosi[pi]e arabinoside, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (L-asparaginase), etoposide, and interferon a-2a and 2b.

Antiviral agents may also be included in the topical foam composition of the present invention along with the rifaximin which include, but are not limited to, acyclovir, amantadine, azidothymidine, ribavirin and vidarabine.

In a case, where pain in a component of the target disorder, the other therapeutic agent can be an analgesic. Useful analgesics include, but are not limited to, phenacetin, butacetin, acetaminophen, nefopam, acetoamidoquinone, and mixtures thereof.

Optionally, a topical anesthetic may also be present in the composition of the invention. For instance, the topical anesthetic may include, but are not limited to dibucaine, lidocaine, pramoxine, benzocaine, tetracaine. In general, the topical anesthetic may be present in any amount which is effective.

In a preferred embodiment, the present invention relates to a pharmaceutical combination product comprising rifaximin adapted for delivery to the colon and/or rectum and a compound selected from, but Snot limited, one or more of 5-acetyl salicylic acid (5-ASA), sulphasalazine, asalazine, prednisolone, or budesonide for simultaneous, separate, or sequential administration.

The topical foam composition, according to the present invention, is usually packed in a suitable pressurized dispensing canister of the aerosol type well known in the art such as an aluminium canister. Each canister is sealed with a suitable foam dispensing valve. Any valve or nozzle/valve assembly which provides a means for releasing the foam from the container and provides foam which is suitable for use in the present invention may be used. The foam that is formed from the composition of the present invention has superior properties. The advantages associated with the topical foam composition according to the present invention is that better results may be obtained in combating the disease and either a lower dosage of the active ingredient or less dosages per day may be necessary to obtain similar results when compared with prior art compositions. For instance, the increased spreading of the foam together with the longer exposure time to the active will result in optimal local effect at the target site. Also, the foam of the present invention may not cause extra irritation of the inflamed target mucosa due to the absence of mineral oils as present in the prior art compositions. Due to these superior properties of the foam, the current invention may represent a valuable alternative to previously known medicines used for the treatment of rectal diseases.

The topical foam composition, of the present invention, is presented in a suitable dispensing container, for example an aluminium aerosol container, fitted with a suitable metered or un-metered valve. Such containers are well known in the art. Where desired, the container can be fitted or supplied together with an applicator device for insertion into the rectum to ensure more efficient administration of the foam.

The dispensing container may be in the form of coated aluminium cans to prevent corrosion, such as epoxy-coated cans. At the time of application, the mixing of the ingredients with propellant may be insured by shaking, optionally with the aid of a mixing bead. The can may be arranged for either “upside down” spraying with the valve at the bottom, or the can have a dip tube so that the foam can be sprayed while the can is upright with the valve at the top.

During the use, the dispensing valve of the can allows rapid expansion of the propellant, which triggers and enhances the foaming action of the surfactant, which thus emerges to entrain the medicated liquid in the form of foam.

The propellant expansion energy is absorbed mainly in forming the foam, thus allowing rectal application without risk.

According to the present invention, the foam may be generated at the moment of therapeutic application.

The topical foam composition of the present invention is applied proximate or to the affected area of the external anus or distal anal canal of the subject.

On administering such compositions, it is sufficient to obtain foams of medium consistency, with a minimum volume of 0.5 g to 10 g of foam introduced into the rectum.

The present invention further provides a process of manufacturing the topical foam composition of rifaximin comprising rifaximin in a nanosize range.

According to another embodiment of the present invention the topical foam composition of rifaximin comprising rifaximin in a nanosize range can be made by

(1) Heating the mixture of emulsifying wax, emulsifier, with surfactant and preservative-water separately.
(2) Adding water soluble alkanol to the preservative-water solution and then mixing with oily phase of step (1)
(3) Adding nano milled rifaximin to the above mixture under stirring and adjusting the required pH using a suitable pH adjusting agent.
(4) Making up the volume of the mixture by adding purified water and finally filling the blend in metal cans and charging the can with propellant.

According to a preferred embodiment of the present invention, Rifaximin can be reduced to a nanosize range by (a) Homogenizing the dispersion of drug, surfactant along with pharmaceutically acceptable carriers (b) Nanomilling the homogenized dispersion obtained in step (a)

It will be appreciated by the person skilled in the art that the topical foam composition comprising rifaximin may further comprise one or more pharmaceutical excipients, selected from, but which are not limited to, emollients or humectants, pH adjusting agents, emulsifiers, foaming agents, fatty alcohols, preservatives, chelating agents, antioxidants, suspending agents, thickening agents, permeation enhancers, occlusive agents, colorants and fragrances or combinations thereof.

Suitable pH adjusting agents, may be selected from, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

In a preferred embodiment, the topical foam composition according to the present invention comprises a suitable pH adjusting agent to adjust the pH in the range from approximately 4 to 8.

Emulsifying waxes, which can be used in the topical foam composition of the present invention, are non-ionic emulsifying waxes such as those described in the U.S. National Formulary (USNF) and ‘Martindale’. An emulsifying wax may be incorporated in the topical composition of the present invention in order to stiffen the foam. The amount of emulsifying wax in the composition is preferably from 1% to 10% w/w based on the total weight of the composition.

Surfactants, which may be employed in the topical foam composition of the present invention, include, but are not limited to fatty alcohols such as, cetyl stearyl, lauryl, myristyl, and palmityl alcohols, surfactants or mixtures thereof.

In another embodiment according to the present invention, a suitable surface active agent can be employed which performs the function of both foaming agent and surfactant.

Suitable emollients and/or humectants, which may be employed in the topical foam composition of the present invention, include, but are not limited to, polyhydric alcohols such as glycols, and polysaccharides, such as ethylene glycol, propylene glycol, butylene glycol, diethylene glycol, dipropylene glycol, glycerin, diglycerin, sorbitol, malvitol, trehalose, raffinose, xylitol, mannitol, polyethylene glycol, propylene glycol, polyglycerin, cholesterol, squaline, fatty acids, octyldodecanol, myristyl alcohol, urea, lanolin, lactic acid, esters such as isopropyl stearate, isopropyl myristate, isopropyl palmitate and isopropyl laurate and the like, preferably myristyl alcohol, octyldodecanol, propylene glycol.

Permeation enhancers may also be incorporated in the topical foam composition of the present invention for delivery of the active ingredient to the mucosal surface. Enhancers which may be employed in the topical foam composition of the present invention include, but are not limited to, sodium glycocholate, sodium taurocholate, polysorbate 80, sodium lauryl sulfate, lauric acid, various alkyl glycosides, dextrins (cyclodextrin, dextran sulfate), fatty acids (phosphatidylcholine, lysophosphatidylcholine), heterocyclic compounds (azone), and small molecules (benzalkonium chloride, cetyltrimethylammonium bromide.

In another preferred embodiment, suitable mucoadhesives may also be employed in the aqueous foam composition of the present invention to improve local retention of mucosally delivered active ingredient.

Mucoadhesive compounds are primarily synthetic or natural polymers that can adhere to the wet mucosal surface. These include synthetic polymers such as, but which are not limited to monomeric alpha cyanoacrylate, polyacrylic acid, hydroxypropyl methylcellulose, and poly methacrylate derivatives. Glue-like polymers include epoxy resins and polyurethanes. Naturally occurring mucoadhesives include chitosan, hyaluronic acid and xanthan gum or mixtures thereof.

Suitable emulsifiers include, but are not limited to, straight chain or branched fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block copolymers, and combinations thereof. One preferred emulsifier is cetyl alcohol. The emulsifier, for example the cetyl alcohol is preferably present in an amount from 0.1 to 5.0% w/w based on the total weight of the composition.

Suitable suspending agents include, but are not limited to, alginic acid, bentonite, carbomer, carboxymethylcellulose and salts thereof, colloidal oatmeal, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

Suitable chelating agents include, but are not limited to, EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphonic acid), 7,19,30-trioxa-1,4,10,13,16,22,27,33-octaazabicyclo[111,11,1]pentatriacon-tane hexahydrobromide, triethylenetetramine-N,N,N′,N″,N′″,N′″-hexaacetic acid, and combinations thereof.

Suitable emollients include, but are not limited to, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof.

Preservatives can be used to prevent the growth of fungi and other microorganisms. Suitable preservatives include, but are not limited to, benzoic acid, sorbic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, thimerosal, and combinations thereof. The preservative is preferably present in an amount from 0.01% to 0.20% w/w, preferably 0.1% to 0.20% w/w, based on the total weight of the composition. In a particular embodiment the composition contains 0.1% to 0.18% w/w methyl paraben and 0.01% to 0.02% w/w propyl paraben.

Examples of suitable antioxidants include, but are not limited to sodium metabisulphite and advantageously this can be used in conjunction with a chelating agent such as a salt of EDTA, e.g. disodium edetate.

The present invention further provides a method of treating, preventing, or alleviating anal disorders comprising administering to a subject in need thereof an effective amount of rifaximin. Anal disorders include one or more of anal fissure, anal ulcer, haemorrhoidal disease, levator spasm, inflammatory bowel disease with anal involvement, irritable bowel syndrome, diarrhea, microbe associated diarrhea, Clostridium difficile associated diarrhea, travelers' diarrhea, small intestinal anal disease, Crohn's disease, chronic pancreatitis, pancreatic insufficiency, colitis, hepatic encephalopathy, or pouchitis.

In a preferred embodiment, the treatment involves contacting or application of the pharmaceutical composition of the present invention to the affected anal area or proximate thereto, such that an effective amount of active ingredient is administered.

In a preferred embodiment, the amount of composition which is employed should be effective for the amelioration, control and/or healing of the anal disease and the prompt and dramatic control or relief of pain resulting from or associated with the disease.

According to yet another embodiment, the anal disorder is or is caused by one or more of anal fissure, anal ulcer, acute hemorrhoidal disease, irritable bowel syndrome, inflammatory bowel disease, (e.g., Crohn's and colitis), travelers' diarrhea, large intestinal anal disease, chronic pancreatitis, pancreatic insufficiency or post-surgical disease (e.g., pouchtis).

In a further embodiment, the effective amount is effective to treat a bacterial infection, e.g., anal diseases including, one or more of anal fissure, anal ulcer, and acute hemorrhoidal disease, irritable bowel syndrome, travelers' diarrhea, small intestinal anal disease, Crohn's disease, chronic pancreatitis, pancreatic insufficiency, colitis, hepatic encephalopathy, antibiotic associated colitis, and/or diverticular disease.

The following examples are for the purpose of illustration of the invention only and are not intended in any way to limit the scope of the present invention.

### Example 1

| Sr. | | Qty/Unit |
|---|---|---|
| No. | Ingredients | (% w/w) |
| 1 | Rifaximin | 5 |
| 2 | Docusate sodium | 0.1 |
| 3 | SLS | 0.3 |
| 4 | Propylene glycol | 20.00 |
| 5 | Emulsifying wax | 1.50 |
| 6 | Cetyl alcohol | 0.18 |
| 7 | Polyoxyethylene 10 stearyl ether | 0.25 |
| 8 | Methyl hydroxybenzoate or Methyl paraben | 0.10 |
| 9 | Propyl hydroxybenzoate or propyl paraben | 0.01 |
| 10 | Triethanolamine | q.s. to pH |
| | | 6.0 |
| 11 | Purified water | q.s. to 100 g |
| 12 | Propellant (Propane/n-Butane/Isobutane) | 4.00 g |
| | Total | 104.00 g |

### Process:

(1) Mixture of emulsifying wax, cetyl alcohol and polyoxyethylene stearyl ether were heated.
(2) Methyl paraben or methyl hydroxybenzoate and propyl paraben or propyl hydroxybenzoate were heated with water.
(3) Propylene glycol was added to the solution of step (2) under homogenization.
(4) Mixture of step (1) was added to the solution of step (3) under homogenization and cooled under stirring.
(5) Rifaximin nanomilled slurry was added to the above mixture and homogenized to cool at room temperature.
(6) A solution of triethanolamine was added to the above mixture for adjusting the pH about 6.
(7) Volume was made up by adding purified water
(8) The blend was filled in metal cans and the can was charged with propellant.

### Example 2

### Non Aqueous Foam

| Sr. No | Ingredients | Qty/unit (% w/w) |
|---|---|---|
| 1. | Rifaximin | 5.00 |
| 2. | Cetostearyl Alcohol | 2.00-8.00 |
| 3. | Triglycerides of capric/caprylic acid | 80.00-95.00 |
| 4. | Propyl paraben | 0.01-0.02 |
| 5. | Butylated hydroxytoluene (BHT) | 0.01-0.1 |
| 6. | Propane/n-butane/iso-butane | 2.00-10.00 |

### Process:

1. Heat part quantity of Triglycerides of capric/caprylic acid, BHT, Propyl paraben and cetostearyl alcohol to about 60-70° C.
2. Homogenize the above mixture for 10 minutes and allow to cool.
3. Separately, heat part quantity of Triglycerides of capric/caprylic acid and rifaximin and homogenize for 10 minutes.
4. Add the above mixture step (3) in the mixture obtained in step (2) maintained at 45° C. under stirring.
5. Cool to room temperature under stirring and fill the prepared blend in aluminium canisters and seal with dispensing valves
6. Charge specified amount of propellant through these valves.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical composition for topical rectal administration in the form of a foam, the composition comprising rifaximin in the form of nanoparticles.

2. A composition according to claim 1, further comprising an aqueous or non-aqueous vehicle.

3. A composition according to claim 2, comprising wherein the non-aqueous vehicle is one or more pharmaceutically acceptably alkanols; one or more pharmaceutically acceptable vegetable oils; or one or more pharmaceutically acceptable organic esters.

4. A composition according to claim 3, wherein the water soluble alkanol is ethanol; propylene glycol; glycerol; polyethylene glycol; polypropylene glycol; propylene glycol; a glyceryl ester; or a mixture thereof.

5. A composition according to claim 2, wherein the vehicle comprises a water soluble alkanol and water, and wherein the w/w ratio of the water soluble alkanol to water from 0.05:10 to 10:0.05.

6. A composition according to claim 2, wherein the vehicle constitutes from 10% w/w to 90% w/w of the total weight of the composition.

7. A composition according to claim 2, wherein the vehicle comprises water in an amount from 20% w/w to 90% w/w of the total weight of the composition, and a water-soluble alkanol in an amount from 0% w/w to 50% w/w of the total weight of the composition.

8. A composition according to claim 1, further comprising at least one surfactant.

9. A composition according to claim 8, wherein the surfactant is present in an amount of from 0.1 to 1.0 w/w of the total weight of the composition.

10. A composition according to claim 1, further comprising at least one propellant.

11. A composition according to claim 10, wherein the propellant is present in an amount from 2 to 20% w/w of the total weight of the composition.

12. A composition according to claim 1, further comprising at least one solubilizer.

13. A composition according to claim 1, further comprising at least one emulsifier.

14. A composition according to claim 13, wherein the emulsifier is present in an amount from 1% to 15% w/w of the total weight of the composition.

15. A composition according to claim 1, further comprising at least one antioxidant.

16. A composition according to claim 1, further comprising at least one preservative.

17. A composition according to claim 16, wherein the preservative is present in an amount from 0.1% to 0.2% w/w of the total weight of the composition.

18. A composition according to claim 1, further comprising a silicone.

19. A composition according claim 1, comprising from 0.01% to 10% w/w rifaximin of the total weight of the composition.

20. A composition according to claim 1, which does not contain any mineral oil.

21. A composition according to claim 1, wherein the composition further comprises 5-acetyl salicylic acid (5-ASA), sulphasalazine, asalazine, prednisolone, or budesonide.

22. A composition according to claim 1, wherein the rifaximin particles having an effective particle size ranging form 10 to 1000 nm.

23. A pharmaceutical composition for topical rectal administration in the form of a foam, the composition comprising the following components in w/w:

| | Quantity |
|---|---|
| Ingredients | % w/w |
| Rifaximin having an effective particle size in | 5 |
| the range 10 to 1000 nm | |
| Docusate sodium | 0.1 |
| SLS | 0.3 |
| Propylene glycol | 20.00 |
| Emulsifying wax | 1.50 |
| Cetyl alcohol | 0.18 |
| Polyoxyethylene 10 stearyl ether | 0.25 |
| Methyl hydroxybenzoate or Methyl paraben | 0.10 |
| Propyl hydroxybenzoate or propyl paraben | 0.01 |
| Triethanolamine | q.s. to pH 6.0 |
| Purified water | q.s. to 100 g |
| Propellant (Propane/n-Butane/Isobutane) | 4.00 g |

24. A pharmaceutical composition as defined in claim 1 1 for administration to the rectum, colon and/or terminal ileum of a patient for the treatment, prophylaxis, or maintenance of remission of a disorder of the rectum, colon, terminal ileum or anus.

25. (canceled)

26. A method of treating, preventing, or alleviating a disorder of the rectum, colon, terminal ileum or anus, comprising administering an effective amount of a pharmaceutical composition according to claim 1 to a subject in need thereof.

27. A process for manufacturing a pharmaceutical composition containing rifaximin, comprising:
(1) heating a mixture of emulsifier and surfactant to form an oily phase;
(2) separately heating a mixture of a preservative and water;
(3) adding a water soluble alkanol to the preservative-water mixture and then mixing with oily phase of step (1); and
(4) Adding rifaximin in the form of nanosized particles to the above mixture under stirring and adjusting the required pH to a desired value using a pH adjusting agent.

28. A process according to claim 27, further comprising heating an emulsifying wax with the emulsifier and surfactant in step (1).

29. A process according to claim 30, further comprising optionally adding purified water to the product of step (4), then filling the product into a dispenser and charging the dispenser with a propellant.

30. A process according to claim 27, wherein the nanosized particles are prepared by reducing the rifaximin to a nanosize range by (a) homogenizing a dispersion of rifaximin with a surfactant in a pharmaceutically acceptable carrier; (b) nanomilling the homogenized dispersion obtained in step (a).

31. A process according to claim 27, wherein the particle size of the rifaximin is less than about 1000 nm.

32. A composition according to claim 1, further comprising a silicone as a lubricant and a stabiliser.

33. A dispenser for a pharmaceutical composition as defined in claim 1, comprising a canister containing under pressure the pharmaceutical composition; a metering valve for measuring a metered dose of the composition from the canister for administration to a patient in need thereof; and an actuator for actuating discharge of the metered dose of the formulation to patient in the form of a foam.

34. A dispenser according to claim 33, wherein the metered dose comprises 0.5 g to 10 g of the pharmaceutical composition.
